SEQUENCE LISTING (6 sequences)

SEQ ID NO: 1 - DNA, 26 bp, artificial
  synthetic primer
gacgagctgtacnnntgctttgccaa

SEQ ID NO: 2 - DNA, 26 bp, artificial
  synthetic primer
ttggcaaagcannngtacagctcgtc

SEQ ID NO: 3 - DNA, 32 bp, artificial
  synthetic primer
gcggcggccatatggaattccagctgaccacc

SEQ ID NO: 4 - DNA, 25 bp, artificial
  synthetic primer
ggcggctgctcttccgcaaccaccn

SEQ ID NO: 5 - DNA, 31 bp, artificial
  synthetic primer
gggtcatgatcacaagtttgtacaaaaaagc

SEQ ID NO: 6 - DNA, 27 bp, artificial
  synthetic primer
ggggatatcaccactttgtacaagaaa

(19) United States
(12) Patent Application Publication
(10) Pub. No.: US 2006/0009623 A1
(43) Pub. Date: Jan. 12, 2006

(54) C-terminal attachment of ligands to proteins for immobilization onto a support

(75) Inventors: Shao Qin Yao, Singapore (SG); Lay Pheng Tan, Singapore (SG); Yee Peng Rina Lue, Singapore (SG)

Correspondence Address: KLARQUIST SPARKMAN, LLP, 121 SW SALMON STREET, SUITE 1600, PORTLAND, OR, 97204, US

(73) Assignee: National University of Singapore

(21) Appl. No.: 10/886,071

(22) Filed: Jul. 6, 2004

(51) Int. Cl. C07K 14/47; G01N 33/53

(52) U.S. Cl. 530/402; 435/7.1

(57) ABSTRACT

The present invention provides methods of immobilizing proteins onto a support, using a cellular expression system or a cell-free expression system, to attach a ligand to the C-terminus of the protein, by an intein-mediated or a puromycin-mediated approach. A method for improving the efficiency of intein-mediated ligand attachment is also provided. The methods of the present invention are useful in the preparation of protein microarrays.

### FIELD OF THE INVENTION

The present invention relates generally to protein arrays and to a method of preparing protein arrays.

### BACKGROUND OF THE INVENTION

In the post-genome era, researchers are faced with the challenge of fully identifying and characterizing all proteins encoded by the human genome. Proteomics is an emerging field aiming to identify and characterize the protein complement of the cell (1). In order to advance the technology and make this field of study realizable, there is a call for the development of high-throughput methods for protein studies. One of the most promising technologies available is the protein microarray, which provides the possibility of simultaneously studying tens of thousands of proteins expressed in a cell or an organism (2).

Despite numerous advances in recent years (2, 3), the development of the protein microarray technology is still in its infancy, facing numerous and complex obstacles, one of which is to develop efficient methods for protein immobilization onto glass surfaces while maintaining their native biological functions (4). This is because proteins are “delicate”—they may unfold and lose their activity if not properly attached to a suitable surface, under conditions that are gentle enough to maintain protein conformation. Hence, the choice of immobilization strategies is a critical determinant for the successful generation of a functional protein microarray.

Currently, few immobilization strategies exist which allow for uniform and stable immobilization of proteins in a microarray (3, 5, 6). Zhu et al. reported the first example of site-specific attachment of (His)₆-tagged proteins onto Ni-NTA-coated glass slides in their generation of the “yeast proteome array” for the yeast Saccharomyces cerevisiae, where more than 90% of proteins encoded by the yeast ORFs were immobilized on a single 25×75 mm glass slide to generate a yeast proteome array (3). A double-tagging system was used to laboriously express proteins in the form of fusions containing both (His)₆ and GST (glutathione-S-transferase) tags, which were then purified on a glutathione column and subsequently immobilized onto a Ni-NTA coated glass slide to generate the proteome array. Generally, arrays in which proteins were site-specifically immobilized (e.g. using (His)₆-Ni-NTA interaction) were found to provide better results than those made with non-specific immobilization methods.

This strategy, however, has a number of drawbacks. First, the entire process is quite tedious, requiring multiple steps of manipulation. Second, protein immobilization using His-tag/Ni-NTA interaction is not strong or robust, limiting the protein array to those downstream applications where mild conditions are used. Third, the use of a macromolecular tag such as GST (MW>25 KDa), which has a moderate affinity for the glutathione resin, may affect the structure and activity of the native protein. Fourth, the use of a GST domain to purify the proteins of interest also limits the strategy to in vitro-based purification methods of proteins expressed in simpler organisms such as yeast, where non-specific background binding of proteins are much lower and thus require only simple, non-stringent washings. Since GST does not bind strongly to its ligand glutathione, it is less likely to withstand the multiple washes often involved in purifying a tagged protein directly from a cell lysate.

Recently, there has been a focus on developing alternative approaches to immobilizing proteins in a microarray in a manner which allows stable, and at the same site-specific, immobilization of proteins (5, 6). Mrksich and co-workers captured cultinase-fused proteins onto glass surfaces coated with a phosphonate ligand, achieving site-specific and covalent immobilization of the proteins (6a). Similarly, by taking advantage of the irreversible alkyl transfer reaction between human O⁶-alkylguanine-DNA alkyltransferase (hAGT) and its substrates, Johnsson et al. successfully developed a site-specific method to covalently immobilize hAGT-fused proteins onto modified glass surfaces (6b). However, both these methods introduce an extra macromolecular tag at the end of protein biotinylation, which may potentially perturbate the protein of interest's conformation, and thus its biological activity.

### SUMMARY OF THE INVENTION

In one aspect, the present invention provides a method of immobilizing a protein onto a support comprising in an expression system, expressing a fusion protein comprising a cleavable intein and reacting the fusion protein with a ligand capable of cleaving the intein to form a protein-ligand; and contacting the products of the expression system with a support that is functionalized with an affinity receptor, thereby immobilizing the protein-ligand onto the support.

In another aspect, the present invention provides a method of increasing the efficiency of intein-mediated covalent attachment of a ligand to the C-terminus of a protein comprising expressing a fusion protein comprising a cleavable intein, wherein the fusion protein comprises at least one small side-chain amino acid immediately upstream to the N-terminus of the intein.

The inventions also provides a method of immobilizing a protein onto a support comprising in a cell-free expression system, expressing a protein and covalently attaching a puromycin-ligand at the C-terminus of the protein; and contacting the products of the cell-free expression system with a support that is functionalized with an affinity receptor, thereby immobilizing the protein onto the support.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic representation of intein-mediated and puromycin-mediated mechanisms of covalently attaching a ligand to the C-terminus of a protein of interest; A is representative of in vitro biotinylation of column-bound proteins, a previously described method; B represents in vivo biotinylation in live cells; and C represents cell-free biotinylation of proteins;

FIG. 2 illustrates specific small molecule-based strategies for site-specific biotinylation of a protein. A. Intein-based, in vitro and in vivo protein biotinylation. B. Puromycin-based protein biotinylation in a cell-free system; C. Biotinylation reagents used in above strategies;

FIG. 3 is an SDS-PAGE gel depicting the influence of C-terminal amino acid residue of the fused protein; A. Proteins bound on chitin beads before MESNA cleavage; B. Proteins remaining on the chitin beads after on-column cysteine-biotin/MESNA cleavage; C. Eluted EGFP (enhanced green fluorescent protein);

FIG. 4 illustrates on column biotinylation of MBP (maltose binding protein); A. Column eluant; B. Eluted MBP incubated with streptavidin magnetic beads;

FIG. 5 illustrates in vitro biotinylation of column-bound proteins; A. Effect of an extra glycine residue on intein-mediated biotinylation; B. Purification and biotinylation of a yeast protein YALOl2W; C. High-throughput expression and biotinylation of yeast proteins;

FIG. 6 A. illustrates Surface Plasmon Resonance analysis of biotinylated MBP on a avidin-functionalized sensor chip; B. SDS-PAGE of purified MBP used in A stained with Coomassie blue;

FIG. 7 illustrates results of bacterial cells expressing MBP-intein incubated with cysteine-biotin/MESNA; A. The clarified cells lysate was analyzed by SDS-PAGE followed by anti-MBP and anti-biotin blot; B. Cell lysate was incubated with streptavidin magnetic beads;

FIG. 8 illustrates in vivo biotinylation of proteins and subsequent protein microarray applications; A. In vivo biotinylation of MBP in E. coli shown by western blots with anti-biotin; B. In vivo biotinylation of yeast proteins (lane 1: YALO12W; lane 2: YGR1S2C) shown by western blots; C. In vivo biotinylation of EGFP in mammalian cells shown by western blots; D. Site-specific immobilization of biotinylated proteins onto avidin slides using bacterial crude lysates;

FIG. 9 illustrates mammalian cells expressing EGFP-intein as analyzed by SDS-PAGE and western blots with anti-EGFP and anti-biotin antibodies;

FIG. 10 is an SDS-PAGE gel that illustrates protein biotinylation in a cell-free system;

FIG. 11 illustrates the efficiency of in vitro protein biotinylation of EGFP fused to three different inteins; a. Yields of fusion proteins and its cleavage efficiency; b.

Yields of eluted/biotinylated EGFP after cysteine/MESNA biotinylation;

FIG. 12 illustrates the efficiency of in vivo protein biotinylation of EGFP fused to three different inteins;

FIG. 13 illustrates puromycin-based site-specific biotinylation of protein in a cell-free system using (a). plasmid DNA, GFP-pIVEX2.4Nde and (b). PCR product as DNA template;

FIG. 14 illustrates efficiency of cell-free biotinylation of green fluorescent proteins (GFP) proteins;

FIG. 15 illustrates the generation of a functional protein array using biotinylated proteins synthesized by the cell-free strategy;

FIG. 16 illustrates (a). a schematic representation of the cloning of destination vector, pDEST-IVEX2.4Nde, suitable for the cell-free biotinylation strategy; and (b). the successful biotinylation of three model proteins cloned into pDEST-IVEX2.4Nde using Gateway™ cloning.

### DETAILED DESCRIPTION

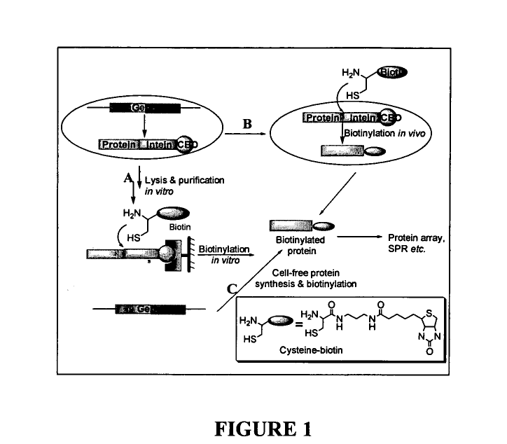

The inventors had previously developed an intein-mediated method of covalently attaching a ligand to the C-terminus of a protein via a peptide bond, for use in the production of protein microarrays, as disclosed in U.S. patent application Ser. No. 10/611,593, which is fully incorporated by reference herein. The attachment at the C-terminus via a peptide bond, preferably of a small molecule such as biotin, results in an increased retention of protein conformation and therefore an increased likelihood of maintained activity of the protein when affixed to a solid support. As described in U.S. patent application Ser. No. 10/611,593, the ligand, for example cysteine-biotin, is attached to a fusion protein bound to an affinity column, and the protein is simultaneously purified in a single step (see FIG. 1, mechanism A). The resulting protein-ligand can then be immobilized to a support that has been functionalised with an affinity receptor that binds the ligand. The biotin-avidin affinity interaction is preferred as it is extremely high affinity and robust. The inventors have now discovered ease and efficiency of covalent attachment of a ligand to the C-terminus of a protein of interest may be increased by modifying the intein sequence, thereby increasing the ease and efficiency of preparing a large number of proteins for use in a protein array. The inventors have also shown that the protein-ligand may be formed in the expression system in which the intein fusion protein is expressed and directly immobilized onto a support, thereby avoiding the step of purification.

Cloning and expression of a large number of proteins of interest can be labour intensive in that it requires multiple steps, some of which can be automated or done in series, but some of which are specific to a particular protein and which therefore must be performed individually. In order to readily handle and process the number of expressed proteins required for a proteomics study, the cloning, expression and manipulation methods need to be streamlined, with as many steps as possible either eliminated or automated. To that end, a method for preparing protein microarrays in which a ligand is covalently attached in vivo to the C-terminus of a fusion protein comprising a cleavable intein can eliminate the purification step. Due to the specificity of the reaction, there is very little background biotinylation of non-target proteins, meaning that the cell-lysate may be directly contacted with the affinity-functionalized support, without the need for further purification.

The inventors have also developed a cell-free system for attaching a ligand to the C-terminus of the protein of interest using puromycin. The use of a cell-free system results in a very rapid, high-throughput method for preparing a large number of proteins that may ultimately be used to prepare an array.

The term protein, as used herein, refers to a polymer of amino acids that are linked by peptide bonds, and includes peptides, which generally refers to relatively small amino acid polymers, for example containing about 30 or fewer residues, or about 20 or fewer residues or about 10 or fewer residues. Where appropriate, the term peptide is used to specifically describe such amino acid polymers and to distinguish from larger proteins. A used herein, the term “amino acids” refers to the standard set of genetically encoded amino acids (alanine, cysteine, aspartic acid, glutamic acid, phenylalanine, glycine, histidine, isoleucine, lysine, leucine, methionine, asparagine, proline, glutamine, arginine, serine, threonine, valine, tryptophan and tyrosine), and derivatives thereof. In the context of polypeptides or peptides created by semi-synthetic or chemical methods, the term “amino acid” also refers to all non-natural amino acids, as well as the D-isomers of the genetically encoded amino acids.

“Expressing” a protein refers to the synthesis of a protein or polypeptide by the translation of a RNA template, usually a mRNA, which encodes the protein or polypeptide and may include a transcription step in which a RNA template is transcribed by a RNA polymerase enzyme from a DNA template. The protein may be expressed within any expression system, such as a cell, or within a cell-free system.

The term “expression system” when used in reference to a cell, or the term “cellular expression system”, refers to a cell that is used to express the protein of interest as a recombinant protein, such that gene for the protein of interest as a fusion protein comprising a cleavable intein is operably linked to a promoter suitable for expression within the expression system chosen. For example, the expression system may be selected from procaryotic and eucaryotic hosts. Eucaryotic hosts include yeast cells (e.g., Saccharomyces cerevisiae or Pichia pastoris), mammalian cells (e.g., COS1, NIH3T3, or JEG3 cells), arthropods cells (e.g., Spodoptera frugiperda (SF9) cells), and plant cells. A skilled person will understand how to express the desired protein or protein fragment in an appropriate expression system. For a protein that is not post-translationally modified and is expected to be soluble, a bacterial expression system may be preferred. However, for large proteins, proteins that are post-translationally modified, or proteins that require mRNA splicing, a eukaryotic system, for example a mammalian system, may be preferred. Commercial sources of cells used for recombinant protein expression also provide instructions for usage of the cells.

When used in reference to a cell-free system, the term “expression system” or “cell-free expression system” refers to an extracellular reaction mixture in which the protein of interest may be expressed and will include the reagents necessary to effect expression of the protein, including ribosomes, tRNAs, amino acids, including amino acyl tRNAs, RNA template, and may further include DNA template, RNA polymerase, ribonucleotides, and any necessary cofactors, buffering agents and salts that are required for enzymatic activity, and may include a cell lysate.

The term “ligand” refers to any ligand that interacts with, for example by binding to, an affinity receptor so as to form a ligand-affinity receptor complex. For example, the ligand may be a small molecule, protein, peptide, lipid or polynucleotide. Preferably, the ligand is a relatively small molecule or moiety, and does not interfere with or interrupt the conformation of the folded protein. The affinity receptor may be any molecule that the ligand interacts with. Any receptor-ligand pair therefore may be suitable and includes biotin-avidin, FLAG-antibody, GST-GSH, MBP-amylose and His-tags-Ni-NTA. Biotin-avidin is particularly preferred due to the strength and stability of the biotin-avidin interaction. Moreover, one skilled in the art will appreciate that certain receptor-ligand pairs may not be suitable, for example if the ligand can have the effect of interfering with the function or structure of the protein that is to be immobilized.

In a first aspect, the present invention provides a method whereby the protein of interest is expressed in an expression system, such as a cellular expression system, as a fusion protein comprising a cleavable intein, as described in U.S. application Ser. No. 10/611,593. A ligand capable of cleaving the intein so as to attach to the C-terminus of the protein is introduced into the cell, and the resulting protein-ligand product of the cellular expression system within the cell lysate is immobilized by directly contacting with an affinity-functionalized support.

The inventors have discovered that there is minimal background binding to the support by the contents of the cell lysate, such that the entire contents of the lysate can be contacted with a functionalized support, resulting in a protein microarray comprising the expressed protein of interest with covalently attached ligand.

In certain embodiments, the cell is a bacterial cell or a mammalian cell, which contains a DNA template encoding the protein of interest. The term “cell” includes a single cell or a plurality of cells, including a population of cells in culture.

The DNA template preferably comprises a gene encoding the protein operably linked to a promoter that is compatible with the particular cell type, and may be a plasmid. For example, the cell may be an E. coli cell, and the DNA template contains a gene encoding the protein of interest operably linked to all of the necessary regulatory sequences such that the gene is transcribed and the RNA is translated by the E. coli cellular machinery. The expression of the gene encoding the protein of interest may be driven by an inducible promoter such that the expression within the cell may be controlled as desired, so as to maximize expression, for example by synchronizing protein expression with logarithmic growth phase of the cell culture.

Inteins, described in U.S. Pat. Nos. 5,981,182 and 5,834,247, the contents of which are incorporated by reference, are protein sequences embedded within a precursor protein that are removed by protein splicing. These sequences can be used to develop fusion protein expression systems to express and purify desired proteins. The intein may be any intein known in the art, where the intein has been mutated such that it only undergoes the first step in the protein cleavage reaction and requires a free thiol agent to complete the cleavage.

One such expression system which is commercially available from New England Biolabs (NEB) uses an intein from the Saccharomyces cerevisiae VMA gene which is mutated (Sce VMA) so that it only undergoes the first step of protein splicing to form a thioester (IMPACT system, pTYB vectors). A skilled person will readily understand how to express the protein of interest as a protein-intein fusion. The intein splicing reaction is completed by the addition of a free thiol agent that is capable of cleaving the thioester bond that forms at the protein-intein interface.

A ligand is introduced to the cell that is capable of cleaving the protein-intein fusion. The ligand may enter the cell by active transport, or it may be able to diffuse into the cell by permeating the cell membrane.

In order to covalently attach to the C-terminus of the protein of interest by cleaving the intein fusion protein, the ligand has a free thiol group and is capable of forming a thioester bond with the peptide backbone.

In a particular embodiment, the ligand is cysteine-biotin, which has a free thiol capable of splicing the protein-intein. The inventors have discovered that by diffusing cysteine-biotin into a cell, a protein-intein fusion may be cleaved in vivo, such that the protein becomes covalently biotinylated at the C-terminus.

Cysteine-biotin includes any biotin derivative with an N-terminal cysteine (cysteine-biotin) in which the N-terminal cysteine will react with the intein thioester, cleaving the intein, and undergoing a nucleophilic rearrangement to form a peptide bond with the protein. The reaction therefore results in the intein fragment being cleaved from the fusion protein and the protein of interest being biotinylated at the C-terminus. Cysteine-biotin may be prepared by known methods using commercially available reagents, such as Boc- or Fmoc-protected cysteine and biotinyl compounds, for example, biotinylethylenediamine, as starting materials.

In some embodiments, when the ligand is attached in vivo using an intein-mediated reaction, an additional thiol agent may be introduced to effect the transfer of the ligand to the C-terminus of the protein of interest while cleaving the intein portion of the protein-intein fusion. An “additional thiol agent” is a compound having a free, reactive thiol group. The additional thiol agent may also be introduced into the cell, either by permeation or active transport. In one embodiment the additional thiol agent is 2-mercaptoenthanesulfonic acid (MESNA). In different embodiments, the additional thiol may be dithiothreitol or other conventional thiols.

The in vivo protein biotinylation strategy presented herein is useful for high-throughput proteomic applications. Particularly, it may prevent premature cleavage of the intein fusion protein in vivo, thus potentially maximizing the yield of the biotinlyated protein obtained. Furthermore, any excess ligand that is used may be readily removed prior to immobilization by simple washes of the cells prior to lysis. As well, since the cells may be lysed and the crude lysate used in subsequent downstream immobilization applications, there is no need for further purification steps. Non-biotinylated proteins in the cell lysate may be washed away from the support in an efficient and highly parallel fashion, resulting in purified proteins immobilized on the microarray. This is likely due to the rare occurrence of naturally biotinylated proteins in the cell, in combination with the highly specific and strong nature of biotin/avidin interaction, which can withstand extremely stringent washing/purification conditions otherwise impossible with other affinity tags.

In another aspect, the inventors have discovered that the efficiency of attachment of the ligand to the C-terminus of the protein may be increased which may be particularly useful in the context of the previously disclosed method of simultaneous column purification and intein cleavage/ligand attachment, although the discovery also is applicable to the above described in vivo intein-mediated ligand attachment strategy.

Particularly, the amino acid residue at the C-terminus of the protein of interest that is immediately upstream to the protein-intein interface has an effect on the efficiency of the intein-mediated attachment of the ligand. The inventors have found that where a Gly residue is immediately upstream to the intein, premature cleavage of the intein in the absence of ligand is reduced. This effect is also seen, albeit to a lesser extent, with other amino acids having small side-chains, for example, Gln, Ala and Thr, and to a lesser extent, Ser and Pro. However, the increased efficiency of ligand attachment was not observed with Val, Met, Asn, Asp and Glu. The term “small side-chain amino acid” as used herein is therefore a reference to any one of amino acids Ala, Gln, Gly, Pro, Ser and Thr.

Therefore, the invention provides a method of increasing the efficiency of intein-mediated covalent attachment of a ligand to the C-terminus of a protein comprising expressing a fusion protein comprising a cleavable intein, wherein the fusion protein comprises at least one small side-chain amino acid immediately upstream to N-terminus of the intein.

Thus, the protein-intein fusion is constructed such that one or more small side-chain residues, or any combination thereof, are immediately upstream to the intein sequence. A skilled person will readily understand how to design and construct such a fusion construct. While increasing the number of small side-chain amino acids, or any combination thereof, is expected to increase the efficiency, as it will be appreciated by a skilled person, the number of small side-chain amino acids should be such that their presence does not interfere with the native conformation of the protein.

In one embodiment, the protein-intein fusion is constructed such that one or more Ala, Gln, Gly, or Thr residues, or any combination thereof, are immediately upstream to the intein sequence. In a particular embodiment, protein-intein fusion is constructed such that one or more Gly residues are immediately upstream to the intein sequence.

Although the intein may be any mutated intein that only undergoes the first step in the protein cleavage reaction, the inventors have further discovered that when the mini-intein from Mycobacterium xenopi is used, the efficiency of ligand attachment is significantly increased, in some instances by as much as ten-fold.

Thus, in a preferred embodiment the intein is the mini-intein from Mycobacterium xenopi (Mxe). This intein may be used either in the in vivo expression and ligand attachment method described above, or in the on-column cleavage and purification method previously described, including with the above mentioned addition of one or more Gly residues at the fusion interface.

To successfully undertake a proteomics study, it is important that each protein of interest can be successfully expressed in soluble form in the expression reaction in order to successfully attach a ligand useful for immobilizing the protein on an affinity-functionalized support. For certain proteins, numerous problems may arise during in vivo protein expression, including the formation of inclusion bodies. This is especially true when one attempts to express eukaryotic proteins in prokaryotic hosts. Other problems include potential proteolytic degradation of the protein by endogenous proteases, as well as expression of proteins toxic to the host cell. Cell-free protein synthesis provides an attractive alternative for protein expression which may potentially overcome many of these problems, and is well-suited for protein microarray applications since small quantities of proteins generated in cell-free system are sufficient for spotting in a protein array. As well, the method can be easily adopted in 96- and 384-well formats with a conventional PCR machine for potential high-throughput protein synthesis (6).

A skilled person will generally understand the term cell-free expression system. For example, the cell-free expression system comprises a cell lysate, for example E. coli cell lysate, and reagents required for the expression reaction, such as amino acids and DNA template. The DNA template may be a plasmid or may be a linear DNA, for example a PCR amplified product. The DNA template preferably encodes the gene for the protein of interest operably linked to the regulatory signals necessary for transcription and translation by the cell-free expression system.

The cell-free expression system further comprises a ligand for covalent attachment to the C-terminus of the protein once it is expressed.

In one embodiment, the protein is expressed in the cell-free system as an intein fusion protein, and the ligand has a free thiol group, as is necessary for the cleavage of the intein from the protein of interest and simultaneous attachment of the ligand, as discussed above.

The cell-free expression strategy using an intein fusion protein is also amenable to the addition of one or more small side-chain amino acids at the fusion interface as discussed above to increase the efficiency of attachment of the ligand, or to the use of the intein from Mycobacterium xenopi.

In addition to the intein-mediated approach, the cell-free expression strategy may be used with a puromycin-mediated approach. The puromycin-mediated approach site-specifically attaches a puromycin-ligand derivative to the C-terminus of the protein of interest by incorporating a ligand-containing puromycin derivative to the end of newly synthesized protein. Puromycin is an aminonucleoside antibiotic produced by Streptomyces alboniger (7) that resembles the 3′ end of the aminoacyl-tRNA. It therefore competes with the ribosomal protein synthesis by blocking the action of the peptidyl transferase, leading to inhibition of protein synthesis on both prokaryotic and eukaryotic ribosomes (8). It has been found that, at low concentrations, for example, about 0.04 to about 1.0 μM, puromycin and its analogs act as non-inhibitors of the ribosomal protein synthesis, and is incorporated at the C-terminus of the newly synthesized protein (9).

A “puromycin-ligand” is any ligand, as defined above, which is conjugated to puromycin such that the puromycin is still capable of being incorporated into a protein or peptide chain. When the puromycin moiety of a puromycin-ligand is incorporated into a protein or peptide chain at the C-terminus, the protein or peptide thereby becomes labelled with the ligand at its C-terminus.

Thus, the attachment of the ligand is achieved by the incorporation of a puromycin-ligand at the C-terminus of the protein of interest by incorporation of the puromycin into the protein chain during synthesis.

The present invention therefore provides a method of immobilizing a protein onto a support comprising in a cell-free expression system, expressing a protein and covalently attaching a puromycin-ligand at the C-terminus of the protein; and contacting the products of the cell-free expression system with a support that is functionalized with an affinity receptor, thereby immobilizing the protein onto the support.

In a particular embodiment, the puromycin-ligand is 5′-biotin-dc-Pmn.

The puromycin-ligand is typically added to the cell-free expression system at a concentration at which the puromycin is incorporated at the C-terminus of the protein of interest. The concentration of puromycin-ligand should be high enough to allow for incorporation at the C-terminus, but not so high as to inhibit protein synthesis by incorporation at positions other than at the C-terminus. For example, the puromycin-ligand may be added to the cell-free expression system at a concentration of about 0.04 μM to about 100 μM, or about 1 μM to about 30 μM.

The cell-free expression system may be used in combination with cloning strategies which are amenable to high-throughput cloning, such as phage lamda site-specific recombination cloning methods. A skilled person will readily understand such methods. In particular, one such method is the Gateway™ system provided by Invitrogen. The Gateway™ cloning strategy provides perhaps one of the most efficient means for high-throughput cloning and proteomics experiments, in that it routinely obtains nearly 100% cloning efficiency. In addition, once a gene is cloned into the Entry™ Cloning vector of the Gateway™ system, it can be easily recloned, once again with nearly 100% efficiency, into a desired Destination™ vector for expression of proteins in different host systems. Consequently, Gateway™ cloning has become the method of choice for high-throughput proteomics research where a large number of genes are involved (10).

For each of the above methods, in order to immobilize the protein of interest into a micro array, the cell lysate, cell-free expression system, or the column eluant, as the case may be, containing the protein-ligand is contacted with a support that is functionalised with a suitable affinity receptor. The excess components that do not have affinity for the support may then be washed away using a suitable rinse solution that will not interrupt the folding of the protein of interest, such as a buffer. In one embodiment of the invention, the biotinylated protein is immobilized onto a support by contacting the expression reaction containing the biotinylated protein directly with an avidin-functionalized support.

Avidin as the term is used herein broadly refers to avidin, which may be derived from different organisms and includes streptavidin and any avidin modified to increase specificity of binding to biotin. As streptavidin is known to have higher nonspecific binding characteristics, in one embodiment, streptavidin can be used to functionalize a support. Numerous materials are suitable for use as support, for instance, silicon, silica, or quartz.

A support may be affinity receptor-functionalized by covalently or non-covalently binding the affinity receptor to the surface of the support. In one embodiment, the support is avidin-functionalized by covalently or non-covalently binding avidin onto the support using methods known in the art. In one embodiment, avidin is covalently bound to a glass surface by reacting a glass surface with glycidoxypropyl-trimethoxysilane silane and then reacting the resulting epoxy glass with avidin. Additional alternatives may be used to functionalize slides with avidin. For example, biotin may be bound to the surface of a slide as a support for avidin, as described by Falsey (11). Another approach is to functionalize the slides with hydroxysuccinimide prior to covalent attachment of avidin.

Suitable support materials in the preparation of a protein array will be apparent to those skilled in the art and include glass, silicon, silica, quartz, carbon, metals, such as gold, platinum, aluminum, copper, titanium and their alloys.

The protein of interest with covalently attached ligand may be spotted onto an affinity receptor-functionalized support using conventional arraying techniques and equipment. A two-dimensional array is preferred as this arrangement allows for a greater number of proteins to be screened at a single time, and optimizes the spot to surface area ratio on the solid support. Within the array, each spot may contain a different protein of interest, or different spots may contain the same protein of interest, as is required for any particular array. The array may contain proteins of interest that comprise an entire or a partial proteome of a particular cell or organism.

The protein arrays produced by the method of this invention may be used to screen for interactions between the immobilized proteins of interest and one or more protein targets. Protein targets may include proteins (including antibodies, enzymes and receptors), drugs, small molecules, hormones, biological molecules (including lipids) and other specific protein ligands.

The most critical issue in generating a protein array is to ensure that proteins maintain their native activity. Proteins which are immobilized onto a support according to the invention have been shown to retain their native activity. Accordingly, the methods of the present invention is ideally suited for preparing a protein array. Furthermore, a large number of proteins may be prepared in a high-throughput manner for immobilization onto a support by methods as described above, further facilitating the preparation of a protein array.

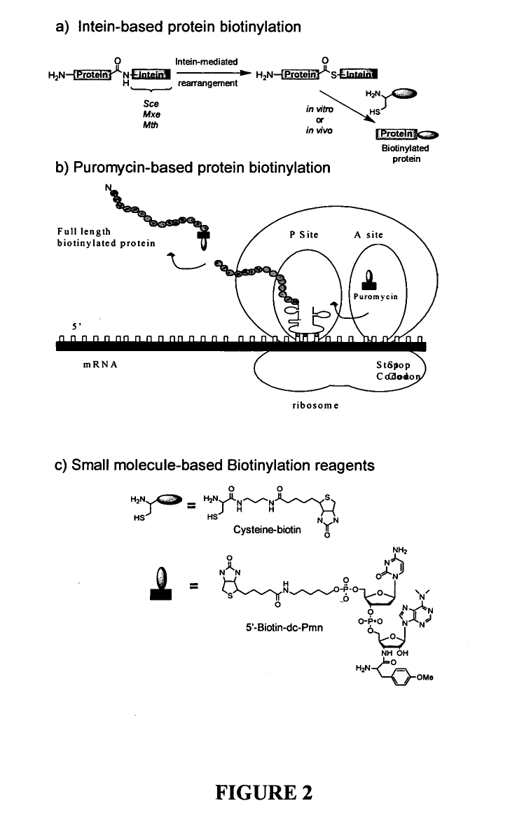

Specifically, the ligand attachment strategies employed in the present invention allow for the covalent attachment of a ligand at the C-terminus of a protein without the requirement of introducing additional amino acids sequences that otherwise may compromise the native protein activity (see FIG. 2A-C). The strategies can avoid tedious protein purification and elution steps, making it possible for proteins in crude lysates to be spotted directly onto a protein array. This enables expression of a large number of ready-to-spot proteins in a high-throughput fashion. In addition, many potential problems associated with recombinant expression, such as protein toxicity to host cells, formation of inclusion bodies and potential protein degradation, can be minimized in the cell-free expression reaction system.

While attachment of biotin to protein has been described, any ligand may be similarly treated to be attached to an intein-fusion protein, or incorporated as a puromycin derivative, to from a protein-ligand that can be immobilized onto a support functionalized with an affinity receptor.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

### EXAMPLES

Materials: Chitin resin, pTYB1, pTYB2, pTWIN1 and pTWIN2 expression vectors were purchased from New England Biolabs (USA). pEGFP expression vector was purchased from Clontech (USA). Cysteine-biotin was prepared as previously described (5a). The puromycin-conjugated biotin, 5′-Biotin-dc-Pmn, was obtained from Dharmacon RNA Technologies (USA). Rapid translation system 100 Escherichia coli HY kit™ and Linear Template Generation Sets™ were purchased from Roche Diagnostics (USA). pT-Rex-DEST30 mammalian expression vector and yeast ex-clones were from Invitrogen (USA). BIAcore X instrument and CMS sensor chip used in SPR experiment were from Biacore (Sweden). MESNA was purchased from Aldrich (USA) or Sigma (USA). Cell beads for cell lysis, avidin, and Dulbecco's modified Eagle's medium (DMEM) basal medium for cell culture were from Sigma. Avidin functionalized glass slides were prepared as described previously. Anti-MBP and anti-OST antibodies were from Santa Cruz Biotechnology (USA). Cy5 dye (λ_{Ex}=633 nm; λₘ=685 nm) was from Amersham Biosciences (USA). FITC dye ((λ_{Ex}=490 nm; (λ_{Em}=528 nm) was from Molecular Probes (USA). Fetal calf serum and antibiotics were from Biological Industries (USA), and tissue culture plates were from Greiner (Germany). Other standard chemicals and biochemicals were purchased from their respective commercial sources, as indicated below.

### Example 1

### Biotinylation of EGFP Mutants Having Different C-Terminal Residues

Methods: pTYB1 and pTYB2 enable expression and isolation of proteins possessing a C-terminal thioester. The target gene is inserted into the polylinker region of each vector, giving rise to the target protein fused in frame to the N terminus of the Sce VMA intein. The only difference between the two vectors lies within the 3′ end restriction site, just before the start of the intein gene. pTYB1 and pTYB2 contains Sap I and Sma I sites at their 3′ ends, respectively. The use of Sap I site in pTYB1 allows the C-terminus of the target protein to be fused directly next to the intein cleavage site, while the use of Sma I site in pTYB2 adds an extra glycine residue to the C-terminus of the target proteins.

All pTYB-1 derived plasmids, including the plasmid coding for the wild-type EGFP fused to an intein, pTYB 1-wEGFP (Lys²³⁹)-intein, were constructed based on NEB's protocols and as previously described (5). The C-terminal residue of wtEGFP in pTYB1-wtEGFP (Lys²³⁹)-intein was site-mutagenized from the original Lys²³⁹ to the other 19 amino acids using QuickChange XL Site-Directed Mutagenesis Kit (Stratagene). Briefly, 19 sets of primers, each containing a primer (5′-GAC GAG CTG TAC NNN TGC TTT GCC AA-3′) [SEQ ID NO:1] and a complementary primer (5′-TT GGC AAA GCA N′N′N′ GTA CAG CTC GTC-3′) [SEQ ID NO:2] were used, in which NNN (and N′N′N′) in each set of primers represents a codon (or anticodon) encoding an amino acid with which Lys²³⁹ in pTYB1-wtEGFP (Lys²³⁹)-intein was replaced.

Upon confirmation by DNA sequencing, the mutated plasmids (e.g. pTYB1-mutEGFP (AA²³⁹)-intein, where AA represents a corresponding mutated amino acid) were transformed into ER2566 E. coli. Protein expression and purification were performed as previously described (5). Briefly, upon harvest and lysis, the clear supernatant was incubated with chitin resin for 30 min at 4° C. with gentle agitation. Subsequently, the resin was washed with the column buffer (20 mM Tris-HCl, pH 8.0, 500 mM NaCl, 1 mM EDTA) followed by incubation with the cleavage buffer (20 mM Tris-HCl, pH 8.0, 500 mM NaCl, 1 mM EDTA, 30 mM MESNA and 1 mM cysteine-biotin) overnight at 4° C.

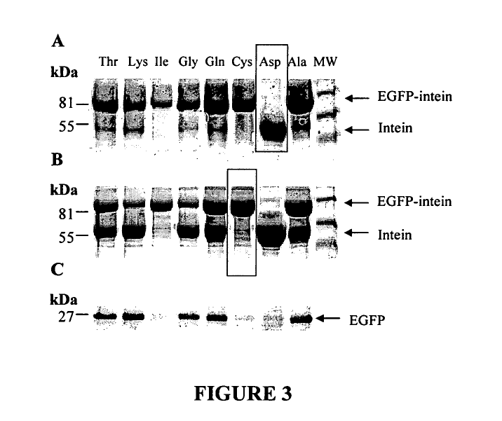

Addition of MESNA was shown previously to promote intein-mediated ligation (12). Upon resin settlement the supernatant which contains the eluted, biotinylated protein was collected and was used directly without further purifications. However, if desired, the eluted fraction may also be passed through a NAP-5 desalting column (Amersham) before use. Resin-bound proteins were analyzed by first boiling the resin with DTT-free SDS-PAGE loading buffer, then separated by SDS-PAGE and stained with Coomassie blue. Premature in vivo cleavage and on-column cleavage/biotinylation of the intein-fusion was determined from the stained SDS-PAGE gel (see FIG. 3).

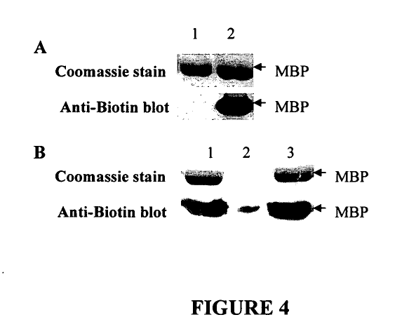

In order to determine the ratio between the biotinylated and the non-biotinylated protein in the eluted fraction, an absorption experiment with streptavidin beads was performed (see FIG. 4; A: lane 1MBP eluted with MESNA only; lane 2, MBP eluted with cysteine-biotin and MESNA; B: lane 1, amount of MBP before streptavidin absorption; lane 2, amount of MBP after streptavidin absorption; Lane 3, MBP bound on streptavidin beads). The eluted fraction was incubated with excessive streptavidin magnetic beads for 1 h at 4° C. to ensure all biotinylated proteins were absorbed onto the beads. Both eluants, before and after streptavidin adsorption, were then analyzed by SDS-PAGE. Western blots with horseradish peroxidase (HRP)-conjugated anti-biotin antibody (from NEB) and the Enhanced ChemiLuminescent (ECL) Plus™ kit (from Amersham) were performed to confirm the presence of biotin-tagged proteins. EGFP (Asp²³⁹)-intein and EGFP (Cys²³⁹)-intein were cloned into pTYB-2 vector via Nde I and Sma I sites based on Impact™-CN protocols (NEB).

Results: The final yield of an in vitro biotinylated protein is primarily dependent upon the amount of the intein fusion recovered from cell extract and its subsequent on-column cleavage/biotinylation efficiency. It was previously reported that the C-terminal amino acid residue of the fused protein at the intein cleavage site greatly affects the cleavage efficiency of the intein (12). In order to design a system in which biotinylation is independent of the C terminus of proteins, we examined the influence of the C-terminal residue of the fused protein on its biotinylation levels. EGFP was cloned into pTYB1 expression vector to generate pTYB1-wtEGFP(Lys 239)-intein, which contains EGFP fused to the intein tag via the original C-terminal residue of EGFP, Lys²³⁹. Site-directed mutagenesis was subsequently performed to mutate Lys²³⁹ to each of the other 19 amino acids. The intein-fused proteins were overexpressed in E. coli, and their in vivo cleavage before cell lysis was assessed. Results are summarized in Table 1. ND=not detected; +=less than 25% cleavage and biotinylation; ++=25-50% cleavage and biotinylation; +++=50-75% cleavage and biotinylation; ++++=75-100% cleavage and biotinylation.

TABLE 1
The influence of C-terminal residues
on biotinylation of EGFP-intein

| | | On column |
|---|---|---|
| C-terminal | In vivo | cleavage and |
| residue | cleavage | biotinylation |
| Ala | + | +++ |
| Arg | +++ | +++ |
| Asn | + | + |
| Asp | 100% | ND |
| Cys | + | ND |
| Gln | + | +++ |
| Glu | ++++ | ND |
| Gly | + | ++++ |
| His | +++ | +++ |
| Ile | + | + |
| Leu | ++ | ++ |
| Lys | ++ | ++++ |
| Met | ++ | +++ |
| Phe | ++ | ++++ |
| Pro | + | ++ |
| Ser | + | ++ |
| Thr | + | +++ |
| Trp | ++ | +++ |
| Tyr | +++ | +++ |
| Val | + | + |

SDS-PAGE analysis showed that acidic amino acids (e.g. Asp and Glu) at the C-terminus of EGFP caused almost complete pre-mature cleavage (˜100%) of the EGFP-intein fusion protein inside the bacteria, while some other residues (e.g. Arg, His and Tyr) caused substantial in vivo cleavage (>50%). The majority of C-terminal residues, however, caused less in vivo cleavage (<50%), thus allowing sufficient amounts of fusion proteins to be obtained prior to subsequent on-column cleavage/biotinylation. Following cell lysis, the fusion protein was first bound to the chitin resin and their on-column cleavage/biotinylation efficiency was subsequently assessed by incubating the resin-bound protein with cysteine-biotin in the presence of MESNA. By streptavidin adsorption experiments with selected proteins, it was determined that >95% of biotinylated proteins were consistently obtained in the eluted fractions following cysteine-biotin/MESNA treatments. Consequently, the amount of on-column protein cleavage was taken to quantify the relative efficiency of protein biotinylation for respective EGFP mutants (column 3 in Table 1). Most amino acids substituted at the cleavage site retained relatively high degrees of protein biotinylation (>50%), while some other residues (e.g. Asn, Cys, Ile & Val) generated relatively lesser amounts of biotinylated protein (<25%). No biotinylation was detected for EGFP mutants having Asp, Glu and Cys substituted at the cleavage site of the fusion.

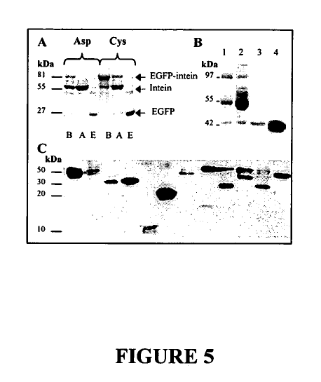

Based on above mutagenesis experiments with the EGFP-intein fusion (Table 1), it was observed that having a Gly residue at the cleavage site minimized the pre-mature cleavage of the fusion in the bacterial cells, and at the same time maximized the subsequent on-column cleavage/biotinylation efficiency. We reasoned that insertion of one or two extra Gly residues at the C terminus of a protein having undesired cleavage-site residues (e.g. Asp & Glu) should optimize protein biotinylation while introducing negligible effect on the protein function. We therefore cloned two EGFP mutants (i.e. EGFP(Asp²³⁹) and EGFP(Cys²³⁹)), containing C-terminal Asp and Cys, respectively, into the pTYB2 vector. The resulting constructs, i.e. pTYB2-EGFP(Asp²³⁹)-intein and pTYB2-EGFP(Cys²³⁹)-intein, were the same as their pTYB-1 counterparts with the addition of an extra Gly at the C-terminus of each mutant. Protein expression from the new constructs revealed that (FIG. 5A; B: proteins bound on chitin beads before cysteine-biotin elution; A: proteins remaining on chitin beads after cysteine-biotin elution; E: eluted biotinylated EGFP), when compared with the original PTYB1 constructs, addition of the extra Gly did substantially lower the in vivo cleavage of the fusion protein (i.e. 70% for pTYB-2 construct vs. 100% for pTYB-1 construct of EGFP(Asp²³⁹)-intein mutant). Significantly improved biotinylation efficiency of the protein was also observed (i.e. up 80% for pTYB-2 construct vs 0% pTYB-1 construct of EGFP(Cys²³⁹)-intein mutant; see FIG. 5A), thereby validating our hypothesis. Consequently, one or two extra Gly residues were introduced in all of our subsequent experiments (vide infra).

### Example 2

### High-Throughput Yeast Protein Expression and Biotinylation

Methods: All high-throughput yeast work was performed in 96-well formats wherever possible. To construct intein-fused yeast proteins, 96 different yeast genes were first PCR amplified from the yeast ex-clones (Invitrogen), and cloned into pTYB1. A common upstream primer (5′-GC GGC GGC CAT ATG GAA TTC CAG CTG ACC ACC-3′) [SEQ ID NO:3] containing an Nde I site with a translation initiation codon (ATG), and individual downstream primers (5′-GGC GGC TGC TCT TCC GCA ACC ACC N₁₅₋₁₈-3′) [SEQ ID NO:4] containing a Sap I site, were used in the PCR reaction to remove the stop codon and at the same time introduce 2 extra Gly residues to the C-terminus of the yeast gene. A standard PCR mixture (25 μl) contained 2.5 μl of 10× HotStarTaq™ DNA polymerase buffer (Qiagen), 0.2 mM of each dNTPs (NEB), 0.5 μM of each primer, 100 ng of plasmid DNA template and 2 units of HotStarTaq™ DNA polymerase (Qiagen). Amplification was carried out with a DNA Engine™ thermal cycler (MJ Research) at 94° C. for 45 sec, 55° C. for 45 sec and 72° C. for 2 min, for 25 cycles. The PCR products were cloned into pCR2.1-TOPO using TOPO TA cloning kit (Invitrogen) prior to double digestion with Nde I and Sap I (NEB). Digested yeast gene fragments of correct sizes were gel-purified and cloned into the pTYB 1 vector via Nde I and Sap I sites to yield intein-fused constructs with two additional Gly residues at the cleavage site.

Upon confirmation by DNA sequencing, the resulting plasmids were transformed into ER2566 E. coli. (NEB), grown in Luria Bertani (LB) medium supplemented with 100 μg/ml of ampicillin at 37° C. in a 250 rpm shaker to an OD600 of 0.6, then induced overnight at room temperature using 0.3 mM isopropyl thiogalactosidase (IPTG). Upon harvest (4000 rpm, 15 mm, 4° C.), cells were resuspended in lysis buffer (20 mM Tris-HCl, pH 8.0, 0.5 M NaCl, 1 mM EDTA, 1% CHAPS, 1 mM TCEP and 1 mM PMSF) and lysed by glass beads (Sigma). The clear lysate was collected by centrifugation, loaded onto microspin columns pre-packed with 100 μl chitin resin and pre-equilibrated with 1 ml of column buffer (20 mM Tris-HCl, pH 8.0, 500 mM NaCl and 1 mM EDTA). To purify the fusion protein, the clear cell lysate was incubated on the column for 30 mm at 4° C. with gentle agitation to ensure maximum protein binding. Unbound impurities were then washed away with 2 ml of column buffer. For biotinylation of yeast proteins, 200 μl of the column buffer containing 100 mM MESNA and 5 mM cysteine-biotin was passed through the column to distribute it evenly throughout the resin before the flow was stopped and the column was incubated at 4° C. overnight. The resulting biotinylated protein was eluted with 100 μl of column buffer, and analyzed on a 15% SDS-PAGE gel. The whole protein expression process was monitored by SDS-PAGE, and the biotinylation of yeast proteins was unambiguously confirmed by Western blots.

Results: To confirm our in vitro biotinylation strategy for potential high-throughput protein expression, we cloned ˜100 different yeast proteins in the form of intein fusions. Yeast proteins were chosen in our studies as their DNA sources are readily available. Two extra Gly residues were conveniently introduced at the C-terminus of each yeast protein by PCR to maximize biotinylation efficiency, and at the same time minimize pre-mature cleavage of the fusion protein in vivo. We found the cloning/protein expression/biotinylation could be readily adopted in 96-well formats, thus enabling high-throughput generation of potentially large numbers of proteins.

Roughly half of the clones (˜50) were further expressed, 31 of which were successfully biotinylated (FIG. 5B & FIG. 5C; FIG. 5B lane 1: proteins bound to chitin beads before cysteine-biotin elution; lane 2: remaining proteins bound to chitin beads after cysteine-biotin elution; lane 3: eluted biotinylated yeast protein; lane 4: immunoblot of lane 3 using anti-biotin antibody; FIG. 5C shows only 12 proteins, the biotinylated fraction on an immunoblot, detected with anti-biotin antibody). The remaining clones (˜20) failed to express as soluble proteins in E. coli and were not pursued further. As shown in FIG. 5B, despite the introduction of 2 extra Gly residues at the C-termini of some yeast proteins, a substantial amount (˜70%) of in vivo cleavage was still observed in the cell lysate, suggesting that alternative approaches may be explored in future to further rectify this problem. Fortunately for most proteins, we were able to isolate sufficient amounts of the fusion proteins. In most cases, subsequent on-column cleavage/biotinylation steps typically eluted the desired biotinylated proteins as the predominant products with acceptable yields (FIGS. 5B and 5C). Varying degrees of protein biotinylation were observed for the yeast proteins (FIG. 5C), which might have been caused by a number of different factors, including differences in the expression level of different yeast proteins, the extent of in vivo self-cleavage and different degrees of on-column cleavage/biotinylation, etc. Of the 31 biotinylated yeast proteins, many are yeast enzymes, covering a wide range of biological activities (i.e. 4 kinases, 4 dehydrogenases, 4 phosphatases, 2 transferases, 2 lyases, I protease, 14 others) and molecular weights (i.e. 10-60 KDa), further validating the generality of our biotinylation strategy.

### Example 3

### Surface Plasmon Resonance Analysis

Methods: All SPR experiments were performed with a BIAcore X instrument. Biotinylated MBP was prepared as described above. Surface activation of the CM5 sensor chip was done using standard amino-coupling procedures according to manufacture's instructions. 1.75 μg of avidin in 10 mM acetate (pH 4.5) and 0.125 M NaCl was passed over the activated chip surface. Excessive reactive groups were then deactivated with 1 M ethanolamine hydrochloride (pH 8.5) before injection of 35 μl biotinylated MBP (10 μg/ml) to the avidin-functionalized surface. Subsequently, 10 μl of anti-MBP antibody (0.1 mg/ml) was injected at a flow rate of 1 μl/min to confirm the immobilization of MBP onto the chip surface. 10 mM HCl was used to regenerate the chip surface before subsequent rounds of antibody injections. The K_{d} of the anti-MBP/MBP binding was determined by BioEvaluation™ software installed on the BIAcore X.

Results: We previously showed that purified biotinylated proteins could be spotted directly onto an avidin-coated glass slide to generate a functional protein array (5). In order to test the stability of the avidin-biotin interaction, and its ability to withstand harsh conditions, we immobilized avidin onto self-assembled monolayers (SAM) and used Surface Plasmon Resonance (SPR) spectroscopy to follow the immobilization of biotinylated proteins onto an avidin-functionalized SAM surface. SPR allows direct visualization of protein immobilization in real time, as well as its subsequent interaction with other proteins (6).

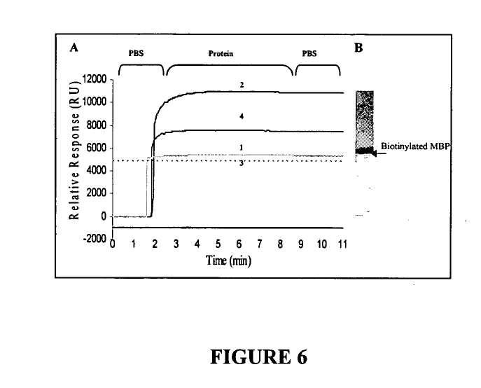

MBP expressed and biotinylated as described earlier (FIG. 6B), was passed over an avidin-functionalized sensor chip having a membrane with associated avidin. The instantaneous interaction of biotinylated MBP with the sensor chip was evident, as shown by a rapid increase in the SPR signal (line I in FIG. 6A). Subsequent washes with PBS did not remove any bound proteins from the chip surface, indicating a stable immobilization of the biotinylated protein to the avidin surface.

To test the real-time interaction of MBP with its binding protein, anti-MBP antibody was flown over the sensor chip: a strong increase in the SPR signal (RU ˜5000) was observed (line 2 in FIG. 6A), indicating specific binding of the antibody to MBP. The dissociation constant (K_{d}) of MBP/anti-MBP binding was estimated from the binding curve to be in the 10⁻¹⁰-10¹¹ M range. A 10 mM HCl solution was subsequently flown over the sensor chip, resulting in the regeneration of the sensor chip while retaining most of the biotinylated MBP on the surface. The slight decrease in the MBP signal (line 3 (dashed) in FIG. 6A) as a result of HCl treatments indicated that some immobilized MBP might have been washed off during the regeneration process. Second-round application of anti-MBP to the regenerated surface again resulted in an increase in SPR signal, albeit with ˜50% of the first-round increase (line 4 in FIG. 6A). Further rounds of regeneration/anti-MBP binding did not appreciably decrease the MBP signal, as well as that from anti-MBP binding (data not shown), indicating the initial decrease in MBP signal was probably due to dissociation of loosely associated MBP to monomeric avidin subunits on the original sensor chip. Once the amount of avidin-bound MBP on the surface was stabilized, further washes of the sensor chip were tolerated. This result is in good agreement with our previous findings that biotinylated proteins immobilized on an avidin-functionalized glass slide were able to withstand extremely harsh washing conditions.

### Example 4

### In Vivo Protein Biotinylation in E. coli

Methods: For in vivo biotinylation of proteins in E. coli, pTYB1 constructs containing MBP and two yeast proteins (YAL012W & YGR152C) were used. Liquid cultures of ER2566 carrying the genes were grown to 0D₆₀₀ of ˜0.6 in LB medium supplemented with 100 μg/ml of ampicillin. Expression of MBP and yeast fusion proteins was induced with 0.3 mM IPTG at 30° C. for 3 h and at room temperature overnight, respectively. MESNA and cysteine-biotin were subsequently added to final concentrations of 30 mM and 3 mM, respectively. Other concentrations of MESNA/cysteine-biotin were also tested but the above conditions gave the best in vivo biotinylation efficiency while maintaining the viability of cells. In vivo biotinylation was allowed to proceed overnight at 4° C. with gentle agitation. Cells were harvested and washed thoroughly with PBS to remove excessive MESNA/cysteine-biotin before being lysed with glass beads. Clear lysates containing the desired biotinylated proteins were collected by centrifugation, and used without further purifications. The entire process was monitored by SDS-PAGE and Western blots.

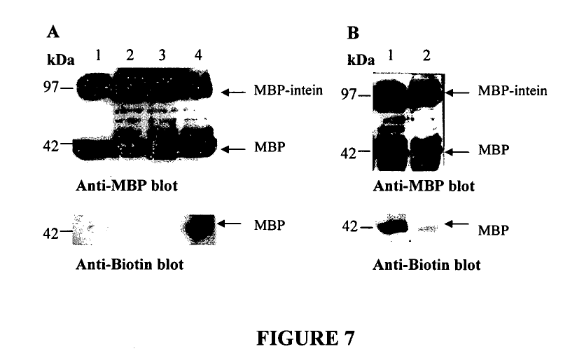

In vivo protein biotinylation was unambiguously confirmed with HRP-conjugated anti-biotin antibody (FIG. 7A; lane 1: lysate of IPTG induced bacterial culture; lane 2: lyaste of bacterial culture incubated with cysteine-biotin only; lane 3: lysate of bacterial culture incubated with MESNA only; lane 4: lysate of bacterial culture incubated with cysteine-biotin/MESNA). Additionally, to confirm the affinity of the in vivo biotinylated protein towards avidin/streptavidin, and to determine the ratio of the biotinylated/non-biotinylated proteins generated in vivo, an absorption experiment with streptavidin beads was performed (see FIG. 7B; lane 1: lysate before streptavidin adsorption; lane 2: lysate after streptavidin adsorption). Briefly, clear cell lysates were incubated with excessive Streptavidin MagneSphere™ Paramagnetic Particles (Promega) at 4° C. for 30 min. The beads were then thoroughly washed with PBS to remove unbound proteins, and subsequently analyzed by boiling in SDS-PAGE loading buffer, then resolved on a 12% SDS-PAGE gel, followed by immunoblotting with HRP-conjugated anti-biotin antibody. Cell lysates before and after streptavidin absorption were also separated on a 12% SDS-PAGE gel followed by Western blots probed with anti-MBP and anti-biotin antibodies.

Results: The intein-mediated biotinylation strategy was extended to living cells. Although intein-mediated protein splicing is part of the naturally occurring processes in cells, its utilities in protein engineering have mostly been limited to in vitro applications (12). Exceptions where in vivo intein-mediated protein splicing have been utilised include the engineering of circular proteins, where head-to-tail native chemical ligation occurred intramolecularly within live cells (13). Also, a recent report by Giriat et al. indicated that intein-mediated protein semi-synthesis was possible in live cells between two designer protein fragments (14). We hypothesized that, if our cysteine-biotin tag is sufficiently cell-permeable, it may be able to cross the membrane of cells overexpressing a desired protein-intein fusion, cleave the fusion and at the same time biotinylate the target protein.

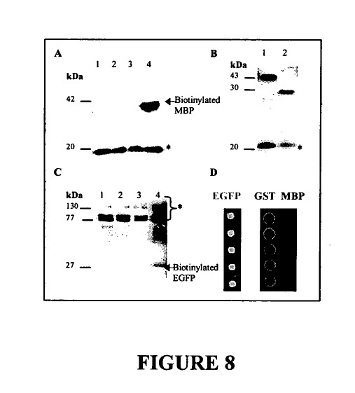

We first tested the in vivo biotinylation of proteins in bacterial cells (FIG. 8A: lane 1: lysate of uninduced bacterial culture; lane 2: lysate of IPTG induced bacterial culture; lane 3: lysate of bacterial culture incubated with MESNA only; lane 4: lysate of bacterial culture incubated with MESNA & cysteine-biotin; and FIG. 8B: lane 1: YALO12W; lane 2: YGR1S2C). It was found that, following IPTG induction to overexpress the intein-fused protein in the growing bacterial cells, the addition of cysteine-biotin/MESNA to the growth media followed by further incubation of the cells resulted in a substantial level of biotinylation in the target protein. Modifications of the cell growth, as well as the in vivo biotinylation conditions, further increased the level of protein biotinylation in the bacterial cells; up to an estimated 20-40% of all MBP expressed was observed to be biotinylated based on streptavidin absorption experiments. Biotinylation was observed in the target protein ONLY if both cysteine-biotin and MESNA were concomitantly added to the cell media (lane 4 in FIG. 8A).

We also showed that proteins from different biological sources (i.e. MBP shown in FIG. 8A and the two yeast proteins shown in FIG. 8B) could be efficiently biotinylated in live bacterial cells.

The purity of the in vivo biotinylated proteins was confirmed by first incubating crude cell lysates with paramagnetic streptavidin beads, then analyzing the bead-bound proteins by SDS-PAGE and Western blotting. In all cases, the desired biotinylated protein could be isolated with high purity. The main impurity detected was acetyl-CoA carboxylase, an endogenous biotinylated protein known in E. coli (* in FIGS. 8A and 8B).

### Example 5

### In Vivo Protein Biotinylation in Mammalian Cells

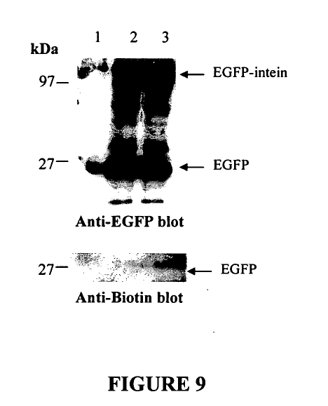

Methods: EGFP-intein was cloned into pTRex-DEST30 (Invitrogen) mammalian expression vector using Gateway™ cloning technology. HEK 293 cells were grown in Dulbecco's modified Eagle's medium (DMEM) supplemented with 10% fetal bovine serum, penicillin (100 units/ml) and streptomycin (100 □g/ml). Cells were seeded at 2.4×106 cells per 100 mm tissue culture plate. After overnight incubation at 37° C., cells were transiently transfected with the vector encoding EGFP-intein using PolyFect™ Transfection Reagent (Qiagen). After 48 h of expression, the culture medium was changed to DMEM containing 10 mM MESNA and 1 mM cysteine-biotin and further incubated at 37° C. overnight. These biotinylation conditions were optimized with respect to cell viability and biotinylation efficiency. Mammalian cells were then harvested, washed thoroughly with PBS to remove excessive biotin, and lysed with glass beads. The entire biotinylation process was monitored by SDS-PAGE and Western blots (with anti-biotin antibody). The biotinylated protein in the mammalian cell lysates was purified using Streptavidin MagneSphere™ Paramagnetic Particles before being unambiguously confirmed by immunoblotting using HRP-conjugated anti-biotin antibody as described earlier (see FIG. 9; lane 1: lysate of untransfected cells; lane 2: lysate of transfected cells; lane 3: lysate of transfected cells incubated with cysteine-biotin/MESNA).

Results: We tested the biotinylation strategy in mammalia cells (FIG. 8C: lane 1: lysate of untransfected cells; lane 2: lysate of transfected cells; lane 3: lysate of transfected cells incubated with MESNA only; lane 4: lysate of transfected cells incubated with MESNA & cysteine-biotin). A mammalian expression vector was constructed such that it contains an EGFP gene fused to the intein. Transient transfection of the construct into HEK 293 cells resulted in a overexpression of green fluorescent proteins inside the cell, which could be readily followed by a UV lamp. Addition of cysteine-biotin/MESNA in basal media containing the transfected cells resulted in appearance of a new biotinylated protein band (FIG. 8C, MW 27 KDa), corresponding to the apparent molecular weight of biotinylated EGFP. In addition, only three other biotinylated proteins were detected, which were also present in untreated cells, which were identified to be the naturally biotinylated proteins pyruvate carboxylase, methylcrotonyl CoA carboxylase and propionyl CoA carboxylase. As shown in FIG. 8C (e.g. * lane 4), the expression of naturally biotinylated proteins appeared to be enhanced with the addition cysteine-biotin. This may be the result of artifacts in our Western blots due to extremely low protein expression level inherent to transient transfection experiments, although we could not completely rule out the possibility that the expression level of endogenous biotinylated proteins was enhanced with the addition of cysteine-biotin/MESNA.

Attempts were also made to quantify the amounts of uncleaved EGFP-intein fusion, self-cleaved protein, as well as properly biotinylated EGFP, by Western blots using anti-EGFP and anti-biotin antibodies. It was found that the majority of the expressed proteins in the mammalian cell lysates were the intein fusion and the self-cleaved product: only a small percentage (˜10%) of proteins expressed were biotinylated (FIG. 9).

### Example 6

### Protein Microarray Generation from Cell Lysates

Methods: All protein microarray work was performed as previously described, with the following modifications. EGFP, GST and MBP were biotinylated in live bacterial cells as described above. 10 ml of bacterial cell cultures were harvested and washed thoroughly with PBS before lysed with 1001 μl of lysis buffer (20 mM Tris-HCl, pH 8.0, 0.5 M NaCl, 1 mM EDTA). The clear cell lysate containing the desired biotinylated protein was spotted directly onto an avidin-functionalized glass slide and subsequently processed as previously described (5). The spotted slides were washed thoroughly with PBST (0.1% Tween in PBS) to remove any non-biotinylated proteins, then incubated with a suitable fluorescently-labeled antibody for 1 hour before washing and scanning with an ArrayWoRx™ microarray scanner (Applied Precision). In order to confirm that the single-step immobilization/purification method removes nonbiotinylated impurities, the crude lysate was first spiked with a pure protein (GST, nonbiotinylated), spotted onto the avidin slide, washed thoroughly and detected with antiGST. As expected, no GST binding was observed on the slide (data not shown).

Results: We examined whether in vivo biotinylated proteins in the crude cell lysate could be used directly for protein microarray applications without a further purification step. We first biotinylated in vivo, as described above, three model proteins (EGFP, GST & MBP). Following cell harvest and lysis, the crude lysates were spotted directly onto avidin-functionalized glass slides, washed and detected either by their native fluorescence (for EGFP) or with FITC-anti-GST and Cy5-anti-MBP, respectively (FIG. 8D). Native fluorescence of EGFP and specific binding between the biotinylated proteins and their corresponding antibodies was observed. Non-biotinylated proteins was not detected on the microarray, as stated above. These results confirmed the binding specificity of biotinylated proteins to the avidin slide and demonstrated that extra purification steps prior to spotting on a protein microarray could be eliminated.

It should be pointed out that one of the major challenges in protein array technologies is the ability of retaining the functional activity of proteins immobilized on the glass surface. In our experiments, the native fluorescence of the immobilized EGFP could be retained on the glass slide for weeks if stored properly at 4° C. (data not shown). Similar results were observed with protein arrays generated using proteins biotinylated in vitro, highlighting the potential of our biotinylation strategies in protein microarray generation.

### Example 7

### Cell-Free Synthesis and Biotinylation of MBP

Methods: The pTYB-1-MBP-intein plasmid was used as the DNA template in the Rapid Translation System (RTS) 100 E. coli HY kit (Roche) for cell-free protein synthesis. Based on the manufacturer's protocol, the reaction was performed at 3° C. for 4 h in a 25 μl reaction with 500 ng DNA as the template. At the end of protein synthesis, MESNA and cysteine-biotin were added to the lysate to final concentrations of 100 mM and 5 mM, respectively, to induce cleavage/biotinylation of MBP at 4° C. overnight. Cell lysates were precipitated with acetone and analyzed by SDS-PAGE. Biotinylation of MBP was unambiguously confirmed by Western blots with HRP-conjugated anti-biotin antibody.

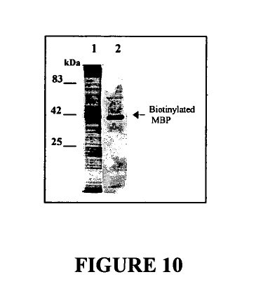

Results: To assess whether our intein-mediated strategy is suitable for biotinylation of proteins expressed in a cell-free system, the MBP plasmid, containing MBP-intein fusion under the transcription control of T7 promoter, was used as the DNA template in a Rapid Translation System (RTS) 100 E. coli HY kit. After cell-free protein synthesis, the reaction was incubated with cysteine-biotin/MESNA, followed by analysis with SDS-PAGE and Western blotting (FIG. 10: lane 1: coomassie stained image of the reaction; lane 2: western blots of lane 1 with anti-biotin antibody). The presence of a 42 kDa band on the anti-biotin immunoblot, and the absence of other bands (FIG. 10, lane 2) indicated successful and exclusive biotinylation of the MBP protein synthesized in the cell-free system.

It should be noted that, among three protein biotinylation strategies presented herein, the cell-free method seems to be the simplest of all. In our hands, however, it is also the least reliable: the efficiency of protein expression as well as the subsequent protein biotinylation depends greatly on a number of different factors, including the nature of the protein itself, the amount and quality of the DNA template used and the kind of cell lysates used for protein expression, etc.

### Example 8

### In Vitro and In Vivo Biotinylation of Proteins with Different Intein Fusions

Methods: All three constructs used in this experiment are otherwise identical, except their inteins. A chitin binding domain (CBD) was fused to the C-terminus of each intein for easy purification of the fusion using chitin columns. The EGFP-Sce VMA intein construct, which contains EGFP fused to the 50 KDa Sce VMA intein from Saccharomyces cerevisiae, was prepared as previously described (5). The EGFP-Mxe intein and EGFP-Mth intein constructs were generated by cloning the EGFP gene (PCR-amplified from pEGFP vector) into pTWIN1 and pTWIN2 vectors, respectively, at the two restriction sites, NdeI and SapI, following protocols provided by the vendor. The resulting constructs, EGFP-Mxe intein and EGFP-Mth intein, contain the EGFP gene fused to the 23 KDa Mxe GyrA mini-intein from Mycobacterium xenopi and the 17 KDa Mth RIR1 mini-intein from Methanobacterium thermoautotrophicum.

All three constructs were transformed into ER2566 E. coli host strain (NEB) for protein expression. Fusion proteins were biotinylated, either in vitro or in vivo, and subsequently assessed for their biotinylation efficiency as previously described (5). Briefly, the transformed ER2566 cells were grown in Luria Bertani (LB) medium supplemented with 100 μg/ml ampicillin at 37° C. in a 250 rpm shaker to an OD₆₀₀ of about 0.5. Protein expression was induced overnight at room temperature using 0.3 mM isopropyl thiogalactosidase (IPTG).

For in vitro-based, on-column biotinylation, cells were harvested and lysed. The resulting lysate was incubated on the chitin column for 30 minutes at 4° C. with gentle agitation. After washing, a column buffer containing 50 mM MESNA and 5 mM cysteine-biotin was added and incubation was continued overnight at 4° C. Elution was done using the elution buffer as previously described (5). Both the eluted and the column-bound fractions were analyzed by SDS-PAGE and Western blots.

For in vivo biotinylation, MESNA and cysteine-biotin (final concentrations: 10 mM and 5 mM, respectively) were added directly to the cell medium following protein expression. The reaction was allowed to proceed overnight at 4° C., after which cells were harvested and washed thoroughly with PBS followed by lysis. The lysate was analyzed directly by SDS-PAGE and Western blots with anti-biotin antibody.

Results: Currently, over 100 different inteins have been identified from different organisms (16). Inteins are believed to have evolved to possess differential protein splicing activities based on the context of their host organisms. We hypothesized that the biotinylation efficiency of a target protein fused to different intein tags in our intein-mediated strategies may differ as well. In our previous studies (5), we successfully used the 50 KDa Sce VMA intein isolated from Saccharomyces cerevisiae to biotinylate proteins, both in vitro and in vivo, with varying degrees of efficiency. We speculated that improved protein biotinylation may be achieved by the use of other intein fusions. We were particularly interested in two naturally occurring mini-inteins, Mxe and Mth, isolated from Mycobacterium xenopi and Methanobacterium thermoautotrophicum, respectively, due to their relatively small sizes (198 and 134 amino acid residues, respectively). Compared with the Sce VMA intein, these two mini-inteins lack the homing endonuclease domain but possess the two important terminal regions which are essential for protein splicing activity. Previous studies indicated that proteins fused to these two mini-inteins undergo splicing efficiently [llc]. We therefore compared, in our intein-mediated strategies, the relative biotinylation efficiency of a protein when fused to each of the three different inteins.

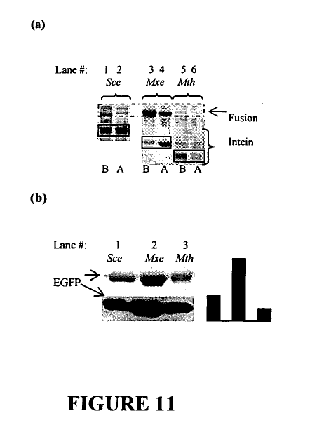

We generated two EGFP-intein constructs, EGFP-Mxe and EGFP-Mth, which express EGFP as the N-terminal fusions of the two mini-inteins, Mxe and Mth, respectively. These constructs were used in experiments together with EGFP-Sce, a construct previously prepared to generate EGFP-Sce VMA intein fusion (5b). All three vectors were transformed into the ER2566 bacterial strain for protein expression. Fusion proteins were extracted, purified on the chitin column, and subsequently cleaved/biotinylated as previously described (5b). The on-column protein biotinylation efficiency was compared by examining (1) EGFP-intein fusions isolated on the chitin column before cleavage, (2) intein tags remained on the column following cysteine-biotin cleavage, as well as (3) the eluted, biotinylated EGFP (FIG. 11a; B: Proteins bound on chitin beads before cysteine-biotin/MESNA cleavage; A: proteins remaining on chitin beads after cysteine-bioitn/MESNA cleavage; FIG. 11b; lane 1: EGFP-Sce intein-CBD fusion; lane 2: EGFP-Mxe intein-CBD fusion; lane 3: EGFP-Mth intein-CBD). In vivo premature cleavage of the protein fusions was evident with both Sce and Mth intein fusions, generating large amounts of EGFP which could not be subsequently biotinylated (FIG. 11a; lanes labeled “3”). This inevitably led to low protein cleavage/biotinylation efficiency upon treatment with cysteine-biotin (lanes 1 and 5 in FIG. 11a; lanes 1 and 3 in FIG. 11b). For the EGFP-Mxe intein fusion however, insignificant in vivo cleavage of the fusion was detected (FIG. 11a, lane 3), with the result that the majority of the expressed EGFP was able to be subsequently biotinylated. As a result, significantly higher overall protein biotinylation efficiency was observed with this intein fusion (FIG. 11b, lane 2 vs lanes 1 and 3), giving rise to an estimated >2-fold increase in the overall protein biotinylation efficiency.

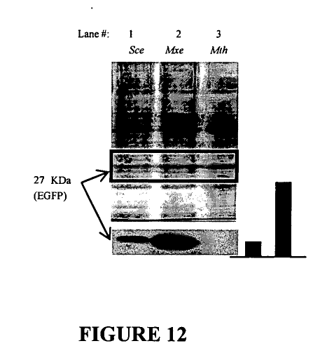

We next assessed the in vivo protein biotinylation efficiency with the three constructs. Cysteine-biotin, together with MESNA, was added to bacterial cells expressing EGFP-Sce intein, EGFP-Mxe intein and EGFP-Mth intein, respectively, and the in vivo biotinylation reaction was incubated further at 4° C. for 24 hrs, as previously described (5). Upon extensive washings, cells were harvested, lysed and directly analyzed by SDS-PAGE and western blots with anti-biotin antibody (FIG. 12; lane 1: EGFP-Sce intein-CBD fusion; lane 2: EGFP-Mxe intein-CBD fusion; lane 3: EGFP-Mth intein-CBD; bar graph: quantification of biotinylation efficiency). Similar to the in vitro experiments described earlier, significantly improved biotinylation efficiency of EGFP was observed with EGFP-Mxe intein (up to 10-fold increase compared with EGFP-Sce VMA; lane 2 vs 1). The other mini-intein fused protein, EGFP-Mth intein, did not produce any significant amount of biotinylated EGFP (i.e. lane 3), indicating that in vivo biotinylation was greatly reduced, presumably as a result of premature cleavage of the fusion.

### Example 9

### Puromycin-Based, Cell-Free Protein Expression and Biotinylation

Methods: The plasmid containing the GFP gene with a (His)₆ tag and under the transcriptional control of the T7 promoter, GFP-pIVEX2.4Nde (Roche), was used as the DNA template in a Rapid Translation System™ (RTS) 100 E. coli HY kit. Each reaction consists of 6 μl of E. coli lysate, 5 μl of reaction mix, 6 μl of amino acids, 0.5 μl of 1 mM methionine, 2.5 μl of the reconstitution buffer. 5′-Biotin-dc-Pmn was added in different concentrations, ranging from 0 μM to 100 μM. The protein synthesis reaction was carried out at 30° C. for 6-9 hours in a DNA Engine™ thermal cycler (MJ Research, USA).

At the end of synthesis, the lysate was analyzed for protein expression and biotinylation with: (1) fluorescence microplate reader (excitation wavelength: 395 nm; emission wavelength: 504 nm) to quantify fluorescence readouts from the expressed GFP (data not shown), and (2) SDS-PAGE analysis and Western blots. Western blots were done with horseradish peroxidase (HRP)-conjugated antibiotin antibody, HRP—conjugated anti-His antibody (NEB) and the Enhanced ChemiLuminescent (ECL) Plus™ Kit (Amersham). The results were used to confirm the degree of GFP expression and biotinylation, respectively, as previously described (5).

The linear template DNA for the RTS reaction was generated with the RTS E. coli Linear Template Generation SetM following the vendor's instructions. Briefly, the PCR mixture (25 μl) contains 2.5 μl of 10× HotStar™ Taq DNA polymerase buffer (Qiagen), 0.2 mM of dNTPs (NEB), 1 μM each of the T7 promoter and terminator primer (Roche), 100 ng of GFP-pIVEX2.4Nde and 2 units of HotStar™ Taq DNA polymerase (Qiagen). Amplification was carried out at 95° C.×1 minute, 60° C.×1 minute and 72° C.×1 minute, for 30 cycles. The resulting PCR-generated, linear template was used directly, without further purifications, in subsequent cell-free transcription/translation/protein biotinylation reactions using conditions similar to those described earlier for the plasmid DNA. Similarly, Western blot analysis with anti-biotin antibody, anti-His antibody and the ECL Plus Kit™ were performed to confirm the presence of GFP expression and biotinylation.

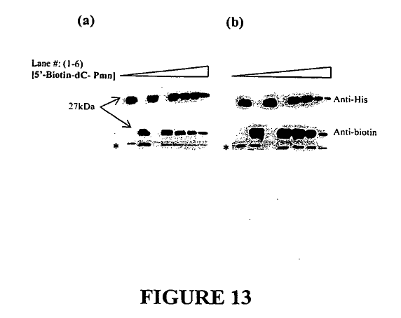

Results: Cell-free reactions were carried out with the plasmid DNA, GFP-pIVEX2.4Nde, as well as its PCR product, both carrying the GFP gene and regulatory elements needed for in vitro transcription/translation, to synthesize biotinylated GFP in the presence of differing amounts of 5′-Biotin-dc-Pmn (FIGS. 13A and 13B, respectively). Western blots with both anti-(His)₆ antibody (top gels) and anti-biotin antibody (bottom gels) were used to determine the overall GFP expression level, as well as the amount of biotinylated GFP produced in each reaction.

As shown in FIG. 13 (top gels), with an increasing concentration of 5′-Biotin-dc-Pmn (O-100 μM), a concomitant decrease in GFP expression was evident, indicating the inhibitory property of puromycin (and its analogs) toward the ribosomal protein synthesis. On the other hand, the amount of biotinylated GFP first increased with increasing concentrations of 5′-Biotin-dc-Pmn (0 to 20/30 μM; lanes 1 to between 3 and 4 in bottom gels of FIG. 13), then gradually decreased (lanes 4 to 6), suggesting that, while a high concentration of 5′-Biotin-dc-Pmn increased the yield of biotin incorporation into GFP, it also inhibited the overall expression of the protein. No other biotinylated proteins were detected in the gels, except acetyl-CoA carboxylase, the only endogeneous biotin-bearing protein present in E. coli (labeled with * in FIG. 13). This result indicates that truncated, biotinylated proteins were not generated during the protein synthesis.

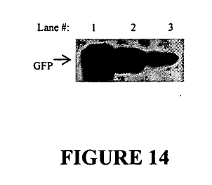

An optimized concentration of 5′-Biotin-dc-Pmn (25 μM in a 25 μl cell-free reaction for RTS™ system) was determined to give the maximum amount of biotinylated GFP from both plasmid and PCR DNA templates. Further optimizations of other parameters (e.g. DNA template concentration, incubation temperature and reaction time) in the cell-free protein biotinylation reaction concluded that the optimum conditions in a 25 μl reaction were the following: 125 ng of DNA template, 25 μM of 5′-Biotin-dc-Pmn, 30° C. for 6-9 hours using the RTS™ system. These conditions were thus used for all subsequent studies, unless indicated otherwise. Control reactions without addition of 5′-Biotin-dc-Pmn were performed (lane I in FIG. 14). No biotinylated GFP was detected in the reaction with either the plasmid or PCR DNA template, confirming that our cell-free protein biotinylation strategy depends entirely on the addition of the puromycin-bearing small molecule.

### Example 10

### Neutravidin Absorption Assay

Methods: To determine the ratio between biotinylated and the non-biotinylated GFP produced in the cell-free system, an absorption experiment with Neutravidin™ beads (Promega) was performed as previously described (5). Briefly, at the end of the cell-free reaction, the lysate containing the biotinylated GFP was incubated with excess Neutravidin beads (prewashed with PBS buffer) for 2 hours at 4° C. with gentle agitation. This ensures all biotinylated GFP present in the lysate was absorbed onto the beads. Both the bead-bound fraction (which contains biotinylated GFP) and the fraction remained in the lysate solution (which contains non-biotinylated GFP) were analyzed by SDS-PAGE and Western blots with anti-EGFP (Clontech) to quantify the percentage of protein biotinylation. Separate blotting experiments with anti-biotin antibody were run in parallel to ensure the successful separation of biotinylated/non-biotinylated GFP in the absorption experiment, as previously described (5).

Results: We investigated the protein biotinylation efficiency in our strategy, by comparing the amount of biotinylated protein synthesized (biotinylated GFP) versus the amount of the total protein synthesized (Biotinylated+non-biotinylated GFP). Taking the cell lysate obtained from the cell-free reaction with the plasmid DNA, GFP-pIVEX2.4Nde, and in the presence of 25 μM 5′-Biotin-dc-Pmn, we subjected it to the Neutravidin™ absorption experiment (5b), in which the biotinylated GFP was separated from non-biotinylated GFP. Upon quantification of the results (FIG. 14; lane 1: lysate before adsorption; lane 2: proteins absorbed onto the beads (biotinylalted fraction); lane 3: lysate remained in solution (non-biotinylated fraction)), more than 50% of GFP was found to be biotinylated (lane 2 vs 3), indicating the relatively reasonable protein biotinylation efficiency provided by this approach.

The RTS™ cell-free system can theoretically yield between 100-500 μg/ml of a protein. In our protein biotinylation system, having taking into account the overall decrease in protein synthesis upon addition of 5′-Biotin-dc-Pmn, we estimated that at least 50% of the total proteins were synthesized, based on theoretical and experimental yield calculations, of which more than 50% were successfully biotinylated. This gave a greater than 25% overall biotinylation yield in our reaction, indicating that between 25-125 μg/ml of the biotinylated protein was produced.

### Example 11

### Protein Microarray Generation from Cell-Free Expression System

Methods: All protein microarray work was performed as previously describe (5), with the following modifications. At the end of the cell-free protein expression/biotinylation using the puromycin method, the lysate (25 μl) was passed through a G25 microspin column (Amersham) to remove most of the residual 5′-Biotin-dc-Pmn. The eluted product (in PBS) was taken, spotted directly onto an avidin-functionalized glass slide and subsequently processed as previously described (5). The spotted slide was washed thoroughly with PBST (0.1% Tween in PBS) to remove any non-biotinylated proteins, then visualized for native GFP fluorescence using an ArrayWoRx™ microarray scanner (Applied Precision, USA). In order to confirm that the single-step immobilization/purification method removes non-biotinylated impurities, the crude lysate was first spiked with a pure protein (GST, non-biotinylated), spotted onto the avidin slide, washed thoroughly and detected with anti-GST. As expected, no GST binding was observed on the slide (data not shown).

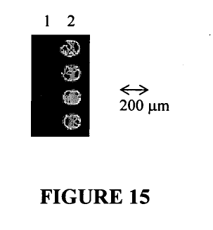

Results: We examined whether biotinylated proteins synthesized using our cell-free system could be used directly for protein microarray applications. We used GFP-pIVEX2.4Nde plasmid as the DNA template, together with 5′-Biotin-dc-Pmn, in a cell-free reaction to generate the biotinylated GFP, as described above. A control lysate was obtained in which GFP was similarly expressed using the same cell-free system but without the addition of 5′-Biotin-dc-Pmn. Upon simple desalting steps following the reaction, the resulting crude lysate, containing newly expressed biotinylated GFP together with other non-biotinylated proteins present in the cell lysate, was taken directly and spotted onto an avidin-functionalized glass side (lane 2 in FIG. 15). The control lysate was treated similarly and subsequently spotted on the same slide (lane 1 in FIG. 15). Native fluorescence of GFP was observed with spots obtained from the biotinylated lysate, but not those obtained from the control lysate (lane 2 vs 1, respectively), indicating the feasibility of using biotinylated proteins synthesized from cell-free systems for protein microarray generation, without the need to purify the proteins of interest away from the remaining reaction mixture prior to generating an array.

### Example 12

### Cell-Free Protein Expression and Biotinylation Using Gateway™ Cloning

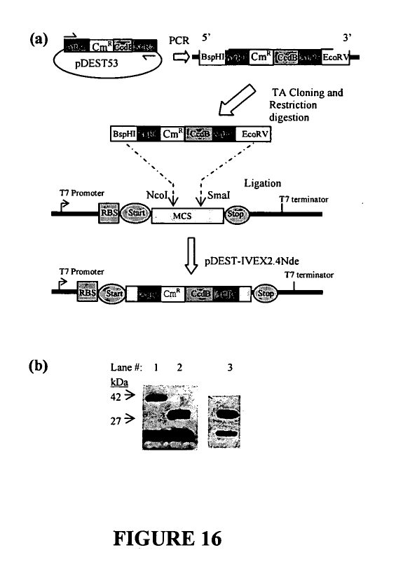

Methods: Attempts to use the Destination vector provided with the Gateway cloning (Invitrogen) in our cell-free protein expression/biotinylation strategy failed, presumably due to the incompatibility between the RTS™ kit and the Gateway Destination vector. We then modified the pIVEX2.4Nde vector, provided with the RTS™ kit, in order to make it compatible with Gateway™ cloning, as follows. To construct the cell-free expression destination vector pDESTIVEX2.4Nde (FIG. 16A), gene fragments containing the attR sites (e.g. attR1 and attR2) flanking the chloramphenicol and CcdB gene were first PCR amplified from pcDNA-DEST 53 (Invitrogen). The upstream primer (5′-GGG TCA TGA TCA CAA GTT TGT ACA AAA AAG C₃′) [SEQ ID NO:5] containing a BspHI site, and the downstream primer (5′-GGG GAT ATCACC ACT TTG TAC AAG AAA-3′) [SEQ ID NO:6] containing an EcoRV site, were used. A standard PCR mixture contained 1× HotStar™ Tag DNA polymerase buffer (Qiagen), 0.2 mM of each dNTPs (NEB), 0.5 μM of each primer, 100 ng of plasmid DNA template and 2 units of HotStar™ Tag DNA polymerase (Qiagen). Amplification was carried out at 94° C.×45 sec, 60° C.×45 sec and 72° C.×2 mm, for 30 cycles. The PCR product was then cloned into pCR2.1-TOPO using TOPO TA cloning kit™ (Invitrogen), following protocols provided by vendor. The resulting TA vector was double digested with BspHI and EcoRV (NEB), gel-purified and cloned into pIVEX2.4Nde at the NcoI and SmaI sites. BspHI and NcoI are isoschizomers that produce compatible cohesive ends while EcoRV and SinaI generate blunt ends. The ligated construct was transformed into DB3.1 E. coli (Invitrogen). Positive clones were selected by the negative selection marker, ccdb, followed by colony PCR and restriction digestion to yield pDEST-IVEX2.4Nde, which can be used as a destination vector for Gateway™ cloning and is compatible with the RTS™ cell-free system. The final expression vectors used for cell-free protein expression/biotinylation, encoding EGFP, MBP and GST genes, respectively, were conveniently constructed by homologous recombination using the above destination vector and following Gateway™ cloning protocols provided by the vendor (Invitrogen). All constructs were confirmed by DNA sequencing.

Results: One of the most essential components in Gateway™ cloning is the Destination vector, in which a target gene is cloned and subsequently expressed in a suitable host. In order to evaluate whether our cell-free protein biotinylation strategy is compatible with Gateway™ cloning, we constructed our own “Destination vector” (FIG. 16a). We inserted the recombinant sites (attR1 and attR2), the chloroamphenicol gene and the CcdB gene into the original vector provided by the RTS™ kit, generating the resulting vector, pDESTIVEX2.4Nde, which is a cell-free expression vector compatible with Gateway™ cloning. In this vector, all regulatory elements (i.e. T7 promoter and terminator, PBS, etc.) needed for cell-free protein expression were already optimized for the RTS™ system, thus compatible with our cell-free protein biotinylation strategy. Three proteins, namely MBP, EGFP, and GST were chosen as models and conveniently cloned into pDEST-IVEX2.4Nde Destination vector, following standard Gateway™ cloning protocols. The resulting constructs were used as DNA templates in our cell-free system to generate the corresponding biotinylated proteins in the presence of 5′-Biotin-dc-Pmn (25 μM).

As shown in FIG. 16b (lane 1: MBP; lane 2: EGFP; lane 3: GST), all three proteins were successfully expressed and biotinylated. The only other biotinylated protein detected in the reaction was acetyl-CoA carboxylase, consistent with our earlier cell-free biotinylation results. From these results, it is possible to combine two high-throughput protein cloning/expression methods, namely the Gateway™ cloning and our puromycin-assisted, cell-free protein biotinylation method. The combination of these high-throughput methods will assist the generation of protein microarrays in a time-efficient manner (i.e. in a matter of hours from DNA to ready-spot proteins) while handling a large number of proteins of interest (i.e. parallel synthesis of many proteins).

As can be understood by one skilled in the art, many modifications to the exemplary embodiments described herein are possible. The invention, rather, is intended to encompass all such modification within its scope, as defined by the claims.

### REFERENCES

1. 1. Chen, G. Y. J., Uttamchandani, M., Lue, R. Y. P., Lesaicherre, M. L. & Yao, S. Q. Curr. Top. Med. Chem. 2003, 3, 705-724.
2. G. MacBeath, S. L. Schreiber, Science 2000, 289, 1760-1763.
3. Zhu, H., Bilgin, M., Bangbam, R., Hall, D., Casamayor, A., Bertone, P., Lan, N., Jansen, R., Bidlingmaier, S., Houfek, T., Mitchell, T., Miller, P., Dean, R. A., Gerstein, M. & Snyder, M. Science 2001, 293, 2101-2105.
4. (a) R. Y. P. Lue, D. S. Y. Yeo, L. P. Tan, M. Uttamchandani, G. Y. J. Chen, S. Q. Yao, in Protein Microarrays (M. Schena, ed), Jones and Bartlett Publishers (Sudbury, MA), 2004, Chapter 2; (b) R. Y. P. Lue, G. Y. J. Chen, Q. Zhu, M. L. Lesaicherre, S. Q. Yao, Methods Mol. Biol. 2004, 264, 85-100.
5. (a) M. L. Lesaicherre, R. Y. P. Lue, G. Y. J. Chen, Q. Zhu, S. Q. Yao, J. Am. Chem. Soc. 2002, 124, 8768-8769; (b) R. Y. P. Lue, G. Y. J. Chen, Y. Hu, Q. Zhu, S. Q. Yac, J. Am. Chem. Soc. 2004, 126, 1055-1062.
6. (a) C. D. Hodneland, Y. S. Lee, D. H. Mi M. Mrksich, Proc. Natl. Acad. Sci. U.S.A. 2002, 99, 5048-5052; (b) M. Kindermann, N. George, N. Johnsson, K. Johnsson, J. Am. Chem. Soc. 2003, 125, 7810-7811.
7. R. A. Lacalle, J. A. Tereero, A. Jimenez, EMBO J. 1992, 11, 785-792.
8. D. Nathans, Proc. Natl. Acad. Sc. U.S.A. 1964, 51, 585-592.
9. (a) E. Miyamoto-Sato, N. Nemoto, K. Kobayashi, H. Yanahawa, Nucleic Acids Res. 2000, 28, 1176-1182; (b) R. W. Roberts, J. W. Szostak, Proc. Natl. Acad. Sc. U.S.A. 1997, 94, 12297-12302.
10. Y. Ho, et al., Nature 2002, 415, 180-183; A. Gavin, et al., Nature 2002, 415, 141-147.
11. Falsey Bioconjugate Chem., 2001, 12:346-353.
12. Xu, M. Q. & Evan, T. C. Methods 2001, 24, 257-277.
13. Scott, C. P., Abel-Santos, E., Wall, M., Wahnon, D. C. & Benkovic, S. J. Proc. Natl. Acad Sc. USA. 1999, 96, 13638-13643.
14. Giriat I., & Muir, T. W. J. Am. Chem. Soc. 2003, 125, 7180-7181.
15. (a) F. B. Perler, Nucleic Acids Res. 1999, 27, 346-347; (b) F. B. Perler, E. O. Davis, G. E. Dean, F. S. Gimble, W. B. Jack, N. Neff, C. J. Noren, J. Thorner, M. Belfort, Nucleic Acids Res. 1994, 22, 1125-1127; (c) M. Q. Xu, T. C. Evan, Methods 2001, 24, 257-277.

What is claimed is:

1. A method of immobilizing a protein onto a support comprising:
in an expression system, expressing a fusion protein comprising a cleavable intein and reacting the fusion protein with a ligand capable of cleaving the intein to form a protein-ligand; and
contacting the products of the expression system with a support that is functionalized with an affinity receptor, thereby immobilizing the protein-ligand onto the support.

2. The method of claim 1 wherein the ligand is biotin and the affinity receptor is avidin.

3. The method of claim 2 wherein the avidin is streptavidin.

4. The method of claim 3 wherein the ligand is cysteine-biotin.

5. The method of claim 4 wherein the fusion protein has one or more Gly residues immediately upstream of the N-terminus of the intein.

6. The method of claim 5 wherein the intein is from Mycobacterium xenopi.

7. The method of claim 6 wherein the expression system is a cell.

8. The method of claim 7 wherein the cell is a bacterial cell.

9. The method of claim 7 wherein the cell is a mammalian cell.

10. The method of claim 7 further comprising introducing an additional thiol agent to the expression system to covalently attach the ligand to the protein.

11. The method of claim 10 wherein the additional thiol agent is 2-mercaptoethanesulfonic acid.

12. The method of claim 6 wherein the expression system is a cell-free expression system.

13. A method of increasing the efficiency of intein-mediated covalent attachment of a ligand to the C-terminus of a protein comprising:
expressing a fusion protein comprising a cleavable intein, wherein the fusion protein comprises at least one small side-chain amino acid immediately upstream to the N-terminus of the intein.

14. The method of claim 13 wherein the small side-chain amino acid is Ala, Gln, Gly, Thr, or a combination thereof.

15. The method of claim 14 wherein the small side-chain amino acid is Gly.

16. The method of claim 15 wherein the intein is from Mycobacterium xenopi.

17. The method of claim 16 wherein the ligand is cysteine-biotin.

18. A method of immobilizing a protein onto a support comprising:
in a cell-free expression system, expressing a protein and covalently attaching a puromycin-ligand at the C-terminus of the protein; and
contacting the products of the cell-free expression system with a support that is functionalized with an affinity receptor, thereby immobilizing the protein onto the support.

19. The method of claim 18 wherein the puromycin ligand is 5′-Biotin-dc-Pmn and the affinity receptor is avidin.

20. The method of claim 19 wherein the avidin is streptavidin.

21. The method of claim 18 wherein the puromycin-ligand is added to the cell-free expression system at a concentration of about 0.04 μM to about 100 μm.

22. The method of claim 21 wherein the puromycin-ligand is added to the cell-free expression system at a concentration of about 1 μM to about 30 μM.
